(19) United States
(12) Patent Application Publication
(10) Pub. No.: US 2015/0265621 A1
(43) Pub. Date: Sep. 24, 2015

(54) Treating Bronchiectasis With Doxofylline and Erdosteine

(71) Applicant: William Wayne Howard, Morristown, NJ (US)

(72) Inventor: William Wayne Howard, Morristown, NJ (US)

(73) Assignee: Alitair Pharmaceuticals, Inc., Morris Plains, NJ (US)

(21) Appl. No.: 14/658,202

(22) Filed: Mar. 15, 2015

Related U.S. Application Data
(60) Provisional application No. 61/967,599, filed on Mar. 22, 2014.

(51) Int. Cl. A61K 31/522; A61K 31/381

(52) U.S. Cl. CPC A61K 31/522; A61K 31/381

(57) ABSTRACT

A method and pharmaceutical composition for the treatment of bronchiectasis comprising doxofylline and erdosteine.

This application claims the benefit of Provisional Application No. 61/967,599 filed Mar. 22, 2014, the disclosures of which are herein incorporated by reference to the extent not incompatible herewith.

### BACKGROUND OF THE INVENTION

1. Field of the Invention

The present invention relates to the synergy provided by simultaneous administration of doxofylline and erdosteine to treat respiratory disorders that are uncommon in the general population and that are often referred to as Orphan Diseases, specifically cystic fibrosis bronchiectasis and non-cystic fibrosis bronchiectasis.

2. Description of the Related Art

In the United States, Orphan Diseases are defined as ones that affect less than 200,000 patients. In regard to respiratory diseases, the following are Orphan Respiratory Diseases of interest to the present invention.
Bronchiectasis associated with Cystic Fibrosis
Non-cystic fibrosis Bronchiectasis
   Cystic Fibrosis Vs. Non-Cystic Fibrosis Bronchiectasis

Cystic fibrosis (CF) is associated with a defective gene that causes the body to produce abnormally thick and sticky mucus. This mucus builds up in the bronchial passages of the lungs and in the body of the pancreas. This build-up of mucus results in life-threatening lung infections and serious digestion problems. Cystic fibrosis is a recessive genetic disorder affecting primarily the lungs, and also the pancreas, liver, and intestine. It is characterized by abnormal transport of chloride and sodium across an epithelium, leading to thick, viscous secretions (Yankaskas et al, 2004).

CF is caused by a mutation in the gene for the protein cystic fibrosis transmembrane conductance regulator (CFTR). This protein is required to regulate the components of sweat, digestive juices, and mucus. CFTR regulates the movement of chloride and sodium ions across epithelial membranes, such as the alveolar epithelia located in the lungs. Although most people without CF have two working copies of the CFTR gene, only one is needed to prevent cystic fibrosis due to the disorder's recessive nature. CF develops when neither gene works normally and therefore has autosomal recessive inheritance (Mitchell et al, 2007).

The name cystic fibrosis refers to the characteristic scarring (fibrosis) and cyst formation within the pancreas, first described by Anderson in 1938. Significantly impaired breathing is the most deleterious symptom of CF. Impaired breathing is a result of frequent lung infections associated with CF. These respiratory infections are treated with antibiotics and other medications. Other symptoms, including sinus infections, poor growth, and infertility affect other parts of the body (Harden, 2004; O'Malley, 2009; Makker, 2009).

In addition to treatment with antibiotics, bronchodilators and anti-inflammatory drugs have also been employed to treat CF (Colombo, 2003; Balfour-Lynn, 2009). Theophylline is a member of the xanthine class of drugs and is known to have both bronchodilator and anti-inflammatory effects (Sullivan et al, 1994). Further, intravenous theophylline has been tested in CF patients and positive results were reported on Forced Expiratory Volume at 1 second (FEV1) and Forced Expiratory Flow at 0.75 seconds (FEF 0.75) (Pan et al, 1989). However, as noted by Colombo, theophylline has received limited use in the treatment of CF due to its high side effect profile and the need for serum monitoring of theophylline. Serum monitoring is needed because theophylline is known as a “narrow therapeutic index” drug i.e. there is a small difference between a therapeutic amount and an amount which causes potentially serious side effects.

Non-Cystic Fibrosis Bronchiectasis

Non-cystic fibrosis bronchiectasis is a disease defined first and foremost by exclusion: i.e. the patient does not have the genetic abnormality that is the sine qua non of cystic fibrosis described above. The patient does, however, have the same symptoms and characteristic lung damage as the cystic fibrosis patient with bronchiectasis, but the cause of the disease is generally unknown. Smith (2011) notes that up to 50% of patients diagnosed with bronchiectasis have no identifiable cause. O'Donnell (2008) states: “Even with exhaustive clinical, laboratory, and pathologic testing, up to 50 to 80% of cases of bronchiectasis may still be idiopathic.”

Bronchiectasis may be present when patients have symptoms such as chronic cough along with daily production of mucus (Smith, 2011). Additional symptoms include dyspnea, hemoptysis, fatigue, and weight loss (Feldman, 2011). Physical findings may be non-specific, but may include crackles and wheezes on lung examination and clubbing of the digits. Pulmonary function testing results generally show airflow obstruction ranging from moderate to severe (O'Donnell, 2008; Feldman, 2011).

The diagnosis of bronchiectasis is confirmed by High-resolution Chest CT (HRCT) scanning, initially described by Naidich et al (1982) and subsequently by Young et al (1992). Ordinary chest X-rays are considered relatively insensitive (Subie and Fitzgerald, 2012). Unlike ordinary chest X-rays, HRCT can detect abnormalities consistent with bronchiectasis such as internal bronchial diameter greater than the diameter of the adjacent bronchial artery, and a lack of bronchial tapering from sequential slices of the bronchi (O'Donnell, 2008).

Feldman (2011) lists the following possible causes of non-cystic fibrosis bronchiectasis: post-infection, immune deficiency, exaggerated immune response, congenital abnormalities, inflammatory pneumonitis, fibrosis, mechanical obstruction and seven other miscellaneous conditions. Feldman does believe that efforts to determine the underlying cause of the disease are important and can affect treatment.

When one reviews the medical literature on the treatment of bronchiectasis, one finds a shocking dearth of well controlled studies for the treatment of bronchiectasis even though the disease was first identified over 200 years ago in 1819 by Laennec (Smith, 2011). For example, ten Hacken and Kerstjens (2011) reviewed twelve potential treatments for bronchiectasis: exercise, prolonged use of antibiotics, anticholinergic therapy, hygiene physical therapy, inhaled corticosteroids, oral corticosteroids, inhaled hyperosmolar agents, leukotriene receptor antagonists, long acting beta2 agonists, oral methyl-xanthines, mucolytics, short-acting beta2 agonists and surgery.

Ten Hacken and Kerstjens list only two treatments as likely to be beneficial for bronchiectasis: exercise and prolonged use of antibiotics. The other ten are listed as “unknown effectiveness”.

Ten Hacken and Kerstjens are not alone in the failure to identify studies that demonstrate effective therapies for bronchiectasis. The following treatments have relatively up to date Cochrane reviews but no randomized controlled trials (RCT) were found so there was nothing to analyze. The Cochrane journal is dedicated to formal, exhaustive reviews of treatments for a wide variety of diseases. For example, Lasserson et al (2011) reported a Cochrane review of the effectiveness of anticholinergic therapy for bronchiectasis. They found NO RCTs evaluating the use of anticholinergic therapy for bronchiectasis. Another Cochrane review for the use of non-steroidal anti-inflammatories for bronchiectasis also found no RCTs in bronchiectasis (Pizzutto et al, 2010). Yet another Cochrane review on the use on oral steroids reported the same score: zero (Lasserson et al, 2001). A Cochrane review of leukotriene receptor antagonists with RCTs found no studies (Corless and Warburton, 2000). A Cochrane Review of long-acting beta agonists by Sheilkh et al (2010) found no RCTs to report. Steele and Greenstone (2010) searched for RCTs using oral methylxanthines in the treatment of bronchiectasis and found none. Likewise, Chang et al (2010) found no RCTs in bronchiectasis to evaluate the effectiveness of influenza vaccine in children and adults. Franco et al 2008 found no RCTs in bronchiectasis to evaluate the effectiveness of short-acting beta2-agonists.

A few treatments with RCTs have been analyzed in Cochran reviews. Wurzel et al (2011) search for RCTs based on short courses of antibiotics. They found one trial that met their criteria. This was one study on nebulized tobramycin conducted at 16 sites with placebo control for four weeks of treatment in adults with CT scan confirmed bronchiectasis. Of 74 enrolled subjects, 60 completed the study. The study did not include any symptom scores but did look at FEV1. There was no effect on FEV1 after four weeks of treatment. There was a significant effect upon eradication of P. aeruginosa.

A Cochran Review of long-term antibiotic treatment by Evans (2011) reported nine RCTs that met their inclusion criteria. While long term antibiotic therapy did reduce bacterial infections there was no statistical effect on exacerbations or FEV1.

Another therapy that was cited in a Cochrane review that had at least one RCT was inhaled cortico-steroids (ICS) (Kapur et al, 2011). In this review Kapur et al found 6 studies that were RCTs. The combined studies demonstrated a positive effect of ICS on FEV1 and FVC but not on the exacerbation rate. However, when the non-placebo controlled studies were eliminated from the analysis, even the FEV1 and FVC effect disappeared. The authors go on to note that there are serious safety concerns with long-term ICS in COPD patients including pneumonia, adrenal insufficiency, osteoporosis and cataracts (Sobieraj et al, 2008).

Positive results were obtained from a combination of a steroid (budesonide) and a LABA (formoterol) in an inhaled dosage form over a 12-month period (Martinez-Garcia et al, 2011). The study group receiving a formoterol-budesonide combination treatment showed a significant improvement, both clinically and statistically on symptoms, dyspnea, number of coughs, and rescue inhaler use. The authors also reported an improvement in HRQL measures. There was a trend to difference in exacerbations in the combination treatment group but that did not reach statistical significance.

This study suffered from several design limitations. First, there was no placebo arm. Second, it was a single site study and blinding of the investigators was questionable. Third, this was a relatively small study with 40 subjects in total. Nonetheless, this is one of the few RCT to show a positive treatment effect for a drug combination that has been widely deployed in other obstructive respiratory diseases such as asthma and COPD.

A major drawback to the use of the combination of an inhaled LABA and an inhaled corticosteroid is the serious safety and compliance issues associated with this therapy. LABAs have a black box warning for death associated with their use, and steroids are known to cause growth retardation in children, osteoporosis in adults, and an increased risk of adrenal insufficiency and pneumonia (Walters, et al 2008), (Allen, 2005), (Sobieraj et al, 2008). Furthermore, inhaled drugs have been associated with poor oral health such as dental caries, candidiasis, ulceration, gingivitis, periodontitis, halitosis and taste changes (Godara, et al 2011). Inhaled therapies also have lower compliance than oral therapies (Price et al, 2011).

In spite of the lack of well controlled studies on the treatment of bronchiectasis, physicians must still treat patients as well as the can. Currently, the treatment of bronchiectasis may involve four paths: antimicrobial therapy, inhaled corticosteroids, airway clearance and surgery (Cheng, 2011). However, it should be noted that there is currently no accepted “best practice” for the treatment of bronchiectasis.

Long term use of antibiotics is aimed at reducing the burden of bacteria and indirectly reduces inflammation. (Feldman, 2011) Likewise, the use of inhaled steroids is also aimed at reducing airway inflammation (Smith, 2011). For example, Tsang et al (2005) demonstrated that therapy with inhaled fluticasone led to clinical improvement in patients who had been treated with 500 ug of inhaled fluticasone twice per day compared to placebo. The effect was especially strong in those infected with Pseudomonas aeruginosa.

The combination of an anti-inflammatory and bronchodilator in treatment of bronchiectasis has been evaluated by Martinez-Garcia and positive results were obtained from a combination of a steroid (budesonide) and a LABA (formoterol) in an inhaled dosage form (Martinez-Garcia et al, 2011).

Airway clearance is considered by some as the most critical component of therapy and may include chest physiotherapy (Feldman, 2011).

Surgery is generally reserved for the most serious cases and when all other therapies have been implemented (Feldman, 2011; O'Donnell, 2008).

Need for Better Bronchiectasis Treatments

The limitations of current treatments strongly suggest that an improved medication for bronchiectasis is very much needed. One possibility is an older drug that combines both bronchodilation and anti-inflammatory effects in one oral drug. Such a drug is theophylline. However, theophylline has serious cardiac, CNS and gastric side effects and is a narrow therapeutic index drug. These side effects severely limit the usefulness of theophylline in the treatment of Orphan Diseases or any disease. The use of theophylline is complicated by its serious, life threatening interactions with various drugs and that it has a narrow therapeutic index, so its use must be monitored to avoid toxicity. It can also cause nausea, diarrhea, increase in heart rate, arrhythmias, CNS excitation (headaches, insomnia, irritability, dizziness and lightheadedness) and death. Its toxicity is increased by erythromycin, cimetidine, and fluoroquinolones, such as ciprofloxacin (package insert, Theo-24). Ten percent of patients treated in an emergency setting for theophylline overdose die (Paloucek et al, 1988). Seizures can also occur in severe cases of toxicity and is considered to be a neurological emergency (Yoshikawa, 2007). Theophylline is a member of a class of drugs known as xanthines and drugs such as caffeine, aminophylline and bromophylline all produce elevated heart rates, CNS simulation and gastric upset. Accordingly, there is an urgent need for a safer drug that combines both bronchodilation and anti-inflammatory effects with a mucolytic to treat bronchiectasis.

A different xanthine that has been shown to have a superior side effect profile as compared to theophylline with fewer CNS, cardiac and gastric side effects is doxofylline. (Page, 2010). In one large, randomized clinical study comparing doxofylline to theophylline in treatment of chronic asthma, 31% of the theophylline subjects dropped out of the study due to side effects but only 11% dropped out of the doxofylline arm due to side effects (Goldstein et al 2002). In this study, doxofylline was as effective as theophylline in treating chronic asthma. The use of doxofylline combined two key treatment results, bronchodilation and anti-inflammatory effects in one composition with a superior side effect and safety profile as compared to either theophylline or the combination of a long acting beta agonist and a steroid.

Given the success of a combination of a bronchodilator and an anti-inflammatory in treating bronchiectasis (Martinez-Garcia et al, 2011), we propose that doxofylline may be an important constituent in a successful treatment for bronchiectasis since doxofylline safely creates bronchodilation and anti-inflammatory effects in one drug.

Additionally as noted by Feldman (2011), “Effective clearance of mucus from the airways is one of the most important, perhaps crucial, treatment modalities that can be instituted in patients with bronchiectasis.” Accordingly, drugs that help with airway clearance would also be considered a priority in the treatment of bronchiectasis. Erdosteine is mucolytic approved for treating COPD and chronic bronchitis in Europe, South America and Asia. In COPD and chronic bronchitis mucus clearance is also an important medical goal.

There are three published studies showing effectiveness of erdosteine in the treatment of bronchiectasis: Crisafulli et al (2007), Buquan and Xinke (2011) and Yipin (2012). The three studies have a total of 170 subjects, 85 of whom received standard care consisting of Percussive Chest Therapy (PCT) for up to 15 days. The remaining 85 received PCT plus treatment with erdosteine 300 mg BID for the same length of time. One should note that these three studies are all open label studies so do not fit the standard of well controlled, randomized, double blind studies.

A comparison of all three studies shows a high degree of correspondence on the objective measures of lung function, FEV1 and FVC. FEV1, FVC, PEF, MIP, MEP, PaO₂ and PaCO₂ measured according to test methods described by Mason et al, 2005. FEV1=forced expiratory volume in one second, measured in liters. FVC=forced vital capacity, total amount air the patient can expel in one breath, measured in liters. PEF=peak expiratory flow, measure in liters per second. MIP=maximum inhalation pressure. MEP=Maximum expiratory pressure. PaO2=partial pressure of oxygen. PaCO2=partial pressure of carbon dioxide. These measures are all obtained with a spirometer that the patient blows into as hard as they can for as long as they can without coughing

Table 1 shows a summary of the three studies with other secondary measures that were collected in all of the studies. Again, the results are similar in terms of order of magnitude and consistent in showing an incremental benefit to adding erdosteine to PCT in bronchiectasis patients.

TABLE I
Change vs Baseline

| | Crisafulli | | Yipin | | Buquan | | Mean | | Wtd Mean Diff | |
|---|---|---|---|---|---|---|---|---|---|---|
| | Erdo | PCT | Erdo | PCT | Erdo | PCT | Erdo | PCT | Erdo | PCT |
| FEV1 L | 0.2 | 0 | 0.34 | 0.03 | 0.19 | −0.01 | 0.2 | 0.0 | 0.26 | 0.01 |
| FVC L | 0.3 | 0 | 0.43 | 0.04 | 0.31 | −0.02 | 0.3 | 0.0 | 0.36 | 0.01 |
| PEF L/s | 0.3 | 0 | 0.4 | 0.1 | 0.3 | 0.1 | 0.3 | 0.1 | 0.35 | 0.08 |
| MIP kPa | 8.9 | 3.9 | 9.9 | 3.1 | 8.1 | 2.4 | 9 | 5.2 | 9.09 | 3.00 |
| MEP Kpa | 8.4 | 9.7 | 11.3 | 3.6 | 8.2 | 9.5 | 9.3 | 7.5 | 9.69 | 6.76 |
| PaO2 mm Hg | 5.1 | 0.3 | 6.3 | 1.2 | 5.2 | 0.6 | 5.5 | 0.7 | 5.70 | 0.83 |
| PaCO2 mm Hg | 0.1 | 0.4 | 2.6 | 0.7 | 0.3 | 0.5 | 1.3 | 0.5 | 1.35 | 0.58 |

| | | | | | | |
|---|---|---|---|---|---|---|
| Num Subs | 15 | 15 | 40 | 40 | 30 | 30 |

| | | | |
|---|---|---|---|
| % Total Subs | 18% | 47% | 35% |

The systematic replication of the Crisafulli study by Buquan and Yipin adds evidence that the use of erdosteine in conjunction with PCT significantly enhances the effect of PCT alone in bronchiectasis patients with mucus hypersecretion. The use of erdosteine with PCT produces significant and clinically meaningful changes in the respiratory functioning of patients with bronchiectasis and mucus hypersecretion as compared to PCT alone.

Summary of Current Bronchiectasis Treatment.

As noted previously, there is currently no generally accepted method for the treatment of bronchiectasis. The history of prior art for the treatment of bronchiectasis indicates that a serious need exists for a treatment that that provides an advancement in the science of bronchiectasis management. Bronchodilators, anti-inflammatory drugs and mucolytics have been used in the treatment of bronchiectasis. However, the prior art does not provide for xanthine based drugs that have a tolerable side effect profile while delivering the twin benefits of bronchodilation and anti-inflammatory effects to treat bronchiectasis. Present treatments combine a bronchodilator and an anti-inflammatory drug to achieve both effects. Further, long acting beta agonists (LABAs) and inhaled steroids both have deleterious health effects associated with them (Walters et al, 2007), (Allen, 2005). Theophylline combines both bronchodilation and anti-inflammatory properties in one oral drug but theophylline is a narrow therapeutic index drug that has serious side effects (Theo-24 Package Insert, Paloucek et al, 1988). These very serious, life threatening side effects and the narrow therapeutic window fundamentally limit theophylline's utility in the treatment of bronchiectasis.

We propose that simultaneous use of doxofylline and erdosteine together will be more successful than either alone because doxofylline is a xanthine and erdosteine is a mucolytic so the two drugs will provide benefits in the treatment of bronchiectasis by two very different and synergistic therapeutic modalities.

It is the object of the present invention to utilize an oral dosage form of doxofylline and erdosteine to treat bronchiectasis as noted above to improve lung function via the synergistic bronchodilation and anti-inflammatory effects of doxofylline and mucolytic effects of erdosteine. Such a pharmaceutical composition comprising doxofylline and erdosteine has never been revealed in the published literature or in issued or pending patents. A demonstration of improved outcome requires a double blind clinical trial wherein each of the drugs is individually tested in treating bronchiectasis patients, along with a treatment regime using both doxofylline and erdosteine together, and a control placebo treatment. We propose that such a clinical trial will show that a composition comprising doxofylline and erdosteine together is more effective in the treatment of bronchiectasis than either drug alone.

Accordingly, the present invention provides novel and useful treatment effects for bronchiectasis patients with an oral xanthine class drug that has a superior side effect profile as compared to theophylline and the mucolytic benefits provided by erdosteine. Simultaneous administration of doxofylline and erdosteine can also overcome the safety and compliance problems associated with delivering a bronchodilator such as a long acting beta agonist or an anti-inflammatory drug such as a steroid via the inhaled route.

### SUMMARY OF THE INVENTION

In one embodiment, the invention is a method for treating a human with a respiratory condition, said method comprising administering to a patient an effective amount of an oral dosage form of pharmaceutical composition comprising doxofylline and erdosteine, said doxofylline and erdosteine in weight proportions ranging respectively from about 5 to 95 to about 95 to 5; said treatment by said pharmaceutical composition providing a synergistic benefit to said patient on a member of the group consisting of lung function measurements of FEV1, FEC, PEF, MIP, MEP, PaO₂ and PaCO₂.

In another embodiment, the invention is a pharmaceutical composition for the treatment of bronchiectasis comprising doxofylline and erdosteine in an oral formulation.

### BRIEF DESCRIPTION OF THE DRAWINGS

FIG. 1 is a schematic diagram illustrating a method of preparation of a doxofylline intermediate to be used in forming a composition of the invention.

FIG. 2 is a schematic diagram illustrating a method of preparation of a erdostein intermediate to be used in forming a composition of the invention.

FIG. 3 is a schematic diagram illustrating a method of preparation of a fixed combination tablet comprising a composition of the invention.

FIG. 4 is a schematic diagram illustrating a method of preparation of a fixed combination capsule comprising a composition of the invention.

### DETAILED DESCRIPTION OF THE INVENTION

In one embodiment, the invention is a method for treating a human with a respiratory condition, said method comprising administering to a patient an effective amount of an oral dosage form of pharmaceutical composition comprising doxofylline and erdosteine, said doxofylline and erdosteine in weight proportions ranging respectively from about 5 to 95 to about 95 to 5; said treatment by said pharmaceutical composition providing a synergistic benefit to said patient on a member of the group consisting of lung function measurements of FEV1, FEC, PEF, MIP, MEP, PaO₂ and PaCO₂.

In another embodiment, the invention is a pharmaceutical composition for the treatment of bronchiectasis comprising doxofylline and erdosteine in an oral formulation

We propose that simultaneous administration of doxofylline and erdosteine will provide five highly desirable and synergistic treatments for bronchiectasis in one oral drug: bronchodilation, anti-inflammatory, mucolytic effects, anti-tussive effects and a synergy with anti-biotics. The proposed method of treating patients having bronchiectasis with doxofylline and erdosteine is safer to use by patients because neither doxofylline nor erdosteine is a narrow therapeutic index drug like theophylline. Furthermore, neither doxofylline nor erdosteine has the serious drug interactions that limit the use of theophylline with many other compounds such as cimetidine or fluoroquinolones. Additionally, both doxofylline and erdosteine are safer than the Long Acting Beta Agonists (LABA) for the treatment of Orphan Respiratory Diseases because LABAs are known to cause death in some patients. Furthermore, neither doxofylline nor erdosteine causes growth retardation in children as does the use of steroids to treat Orphan Respiratory Diseases. Steroids are also known to cause bone loss in adult patients whereas neither doxofylline nor erdosteine are known to have this effect.

The method of treating bronchiectasis with an oral formulation of doxofylline and erdosteine will lead to greater compliance and persistence than inhaled drugs used to treat bronchiectasis. Greater compliance will lead to better health outcomes as compared to treatment methods that result in lower compliance such as the use of inhaled drugs to treat bronchiectasis.

The method of treating bronchiectasis patients with doxofylline and erdosteine can utilize pharmaceutical compositions formulated, for example, as a capsule or compressed tablet or as a liquid or a suspension. Furthermore, the method of the invention may utilize compositions which would deliver either an immediate release effect (IR) or a sustained release effect (SR) or both an IR and SR effect. This will involve the use of an IR component and sustained release (SR) component in the pharmaceutical composition.

In addition, the method of the invention may utilize doxofylline and erdosteine with an additional medicinal component that would constitute a fixed dose combination product. A pharmaceutical composition comprising doxofylline and erdosteine may also be used concomitantly with additional medicinal agents that are useful to treating bronchiectasis such as antibiotics.

By “orphan respiratory disease” is meant any disease primarily affecting the function of the nose, larynx, trachea, lungs, bronchi, alveoli and associated muscles in the respiratory system which facilitate the oxygenation of the blood with a concomitant removal of carbon dioxide and other gaseous metabolic wastes from blood and the disease afflicts less than 200,000 people in the United States.

The term “obstructive lung disease” means any disease that causes the airways of the lungs to become narrow, blocked or impaired so that a patient cannot exhale completely. Because of damage to the lungs or narrowing of the airways inside the lungs, exhaled air comes out more slowly than normal. At the end of a full exhalation, an abnormally high amount of air may still remain in the lungs.

By “orphan obstructive lung disease” is meant any orphan respiratory disease that includes obstructive lung disease.

By “pharmaceutically active agent” is meant agents other than food articles that are intended to diagnose, cure, mitigate, treat or prevent disease in man or other animals or that are intended to affect the structure or any function of the body of man or other animals that are physiologically acceptable. The agent could be a combination of drug therapies as well as a single agent.

By “physiologically acceptable” is meant those substances that are adequately tolerated without causing unacceptable negative side effects.

By “immediate release”/IR is meant that the pharmacologically active agent is released from the formulation immediately such that at least 80%, preferably at least 85%, more preferably at least 90%, or most preferably at least 95% of the agent in the formulation is absorbed into the blood stream of a patient two hours after administration. Whether a pharmaceutical composition is formulated for immediate release can be determined by measuring the pharmacokinetic profile of the formulation.

By “extended release” is meant that the pharmaceutically active agent is released from the formulation at a controlled rate such that the formulation allows for a reduction in dosing frequency as compared to that presented by a conventional dosage form, e.g. an immediate release dosage form.

The invention also includes methods and compositions for delivering combinations of pharmaceutically active compounds. Examples of such combinations are:

A: a composition comprising doxofylline and erdosteine and a steroid

B a composition comprising doxofylline and erdosteine and a long acting beta agonist

C: a composition comprising doxofylline and erdosteine and an anti-cholinergic agent

D: a composition comprising doxofylline and erdosteine and an antibiotic

E: a composition comprising doxofylline and erdosteine and a leukotriene inhibitor

Dosage Forms

Suitable dosage forms include syrups, suspensions, tablets, capsules, granules, powders, pellets and the like.

Solid Oral Dosage Delivery Systems

Suitable delivery systems include compressed tablets, capsules, granules/multi-particle systems, pellets as well as granules/multi-particle systems, pellets filled into capsules and the like

Liquid Oral Dosage Delivery Systems

Suitable delivery systems include solutions and suspensions and the like.

### EXAMPLES

### Example 1

In a double blind study, patients suffering from bronchiectisis are alternatively treated with either doxofylline alone, with erdosteine alone, with the inventive drug composition comprising doxofylline and erdosteine, or with a placebo. One or more objective measures of lung function such as FEV1 and FVC. FEV1, FVC, PEF, MIP, MEP, PaO₂ and PaCO₂ are significantly improved with the inventive drug composition over results with either drug alone.

Each of the pharmaceutical compositions of the examples below is useful for simultaneous oral administration of doxofylline and erdosteine

### Example 2

### IR Dosage Form

### Hard Gelatin Capsule

| doxofylline and erdosteine | 400 mg/300 mg | |
|---|---|---|
| Microcrystalline cellulose | 200 | mg |
| Modified Starch 1500 | 200 | mg |
| Magnesium Stearate | 8 | mg |
| Empty Capsule Shell #0 | 96 | mg |
| Total Dosage Form Weight | 904 | mg |

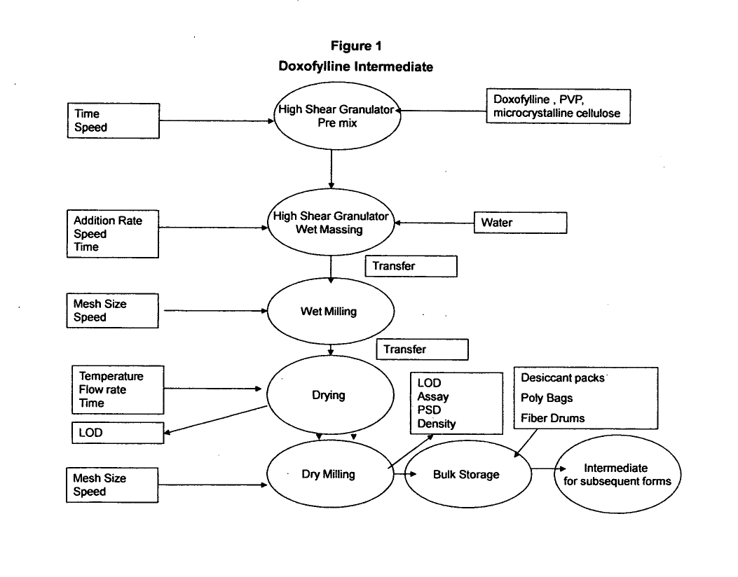

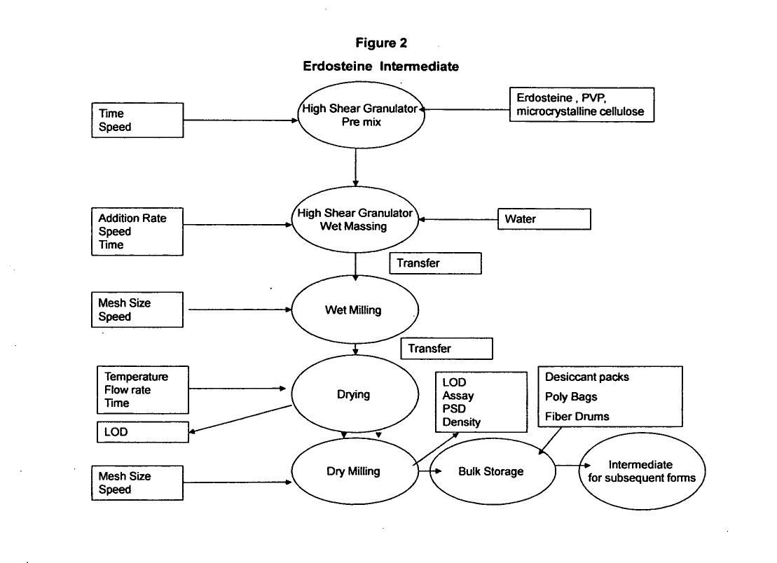

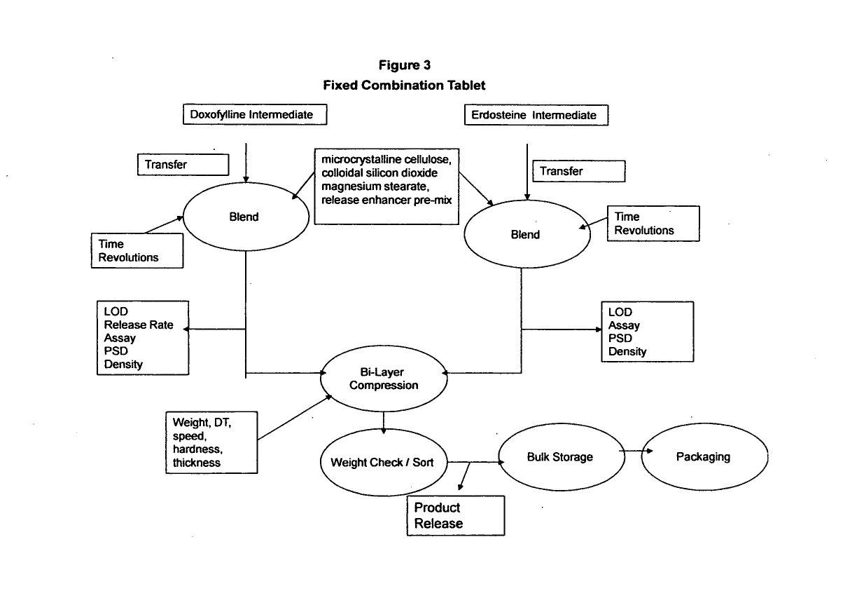

The immediate release capsules are manufactured using a standard wet granulation technique. Doxofylline and erdosteine, microcrystalline cellulose and modified starch 1500 are dry blended in a suitable mixer such as a planetary mixer. Water USP is then added to the dry powder blend while mixing until suitable granules are formed. The wet mass is dried to a level of approximately 1.5% loss on drying (LOD). The dried granules are then screened/milled to a suitable particle size, blended with the magnesium stearate and subsequently filled into hard gelatin capsules having a final filled weight of 904 mg. The dried blend may be tested for assay and content uniformity prior to the encapsulation. The process is shown in FIGS. 1, 2 and 3 below.

### Example 3

### IR Dosage Form

### Compressed Tablet

| doxofylline and erdosteine | 400 mg/300 mg | |
|---|---|---|
| Microcrystalline cellulose | 200 | mg |
| Polyplasdone XL | 20 | mg |
| HPMC 3cps | 25 | mg |
| Anhydrous Lactose | 200 | mg |
| Magnesium stearate | 6 | mg |
| Colloidal Silicon Dioxide | 6 | mg |
| Total Dosage Form Weight | 857 | mg |

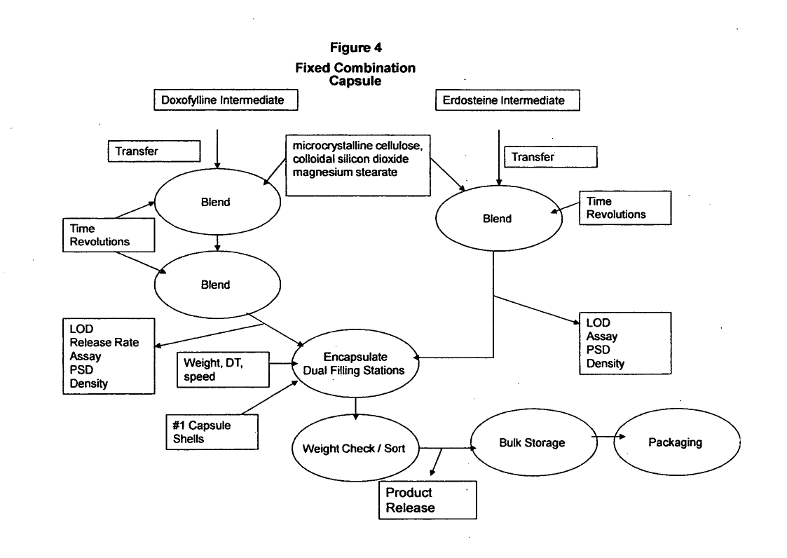

The immediate release tablets are manufactured using a standard wet granulation technique. The doxofylline and erdosteine, microcrystalline cellulose, Polyplasdone XL, and anhydrous lactose are dry blended in a suitable mixer such as a planetary mixer. The HPMC 3 cps is added to a sufficient amount of water USP to form a suspension/solution. This is then added to the dry powder blend while mixing until suitable granules are formed. The wet mass is dried to a level of approximately 1.5% loss on drying (LOD). The dried granules are then screened/milled to a suitable particle size, blended with the magnesium stearate and colloidal silicon dioxide and subsequently compressed into tablets having a final weight of 857 mg. The dried blend may be tested for assay and content uniformity prior to the compression. The process is shown in FIGS. 1, 2 and 4 below.

### Other Embodiments

All publications, patent applications, and patents mentioned in this specification are herein incorporated by reference to the extent not incompatible herewith.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

### REFERENCES

### Articles and Books

ALLEN D B. Inhaled steroids for children: effects on growth, bone, and adrenal function. Endorinol Metab Clin North Am. 2005; 34(3): 555-64.
BALFOUR-LYNN IM, WELCH K. Inhaled corticosteroids for cystic fibrosis. Cochrane Database Syst Rev. 2009 Jan. 2; (1): CD001915.
BUQUAN Z, XINKE L I. Curative effect of treating elderly bronchiectasis and mucus hypersecretion patients. The Journal of Practical Medicine, Vol 27, Issue 6, 2011.
CHANG C C, MORRIS P S, CHANG A B; Influenza vaccine for children and adults with bronchiectasis. Cochrane Database Syst Rev. 2010, Issue 9.
CHENG, G S. Non-cystic Fibrosis Bronchiectasis. PCCSU Volume 25, Lesson 18, 2011. Accessed at www.chestnet.org
Colombo C, Russo M C, Zazzeron L, Romano. Liver disease in cystic fibrosis. J. Pediatr. Gastroenterol. Nutr. 43 Suppl 1: S49-55.
Corless J A, and Warburton C J. Leukotriene receptor antagonists for non-cystic fibrosis bronchiectasis (Review). Cochrane Database of Systematic Reviews, 2011, Issue 7.
Crisafulli, E, Coletti O, Costi S, Zanasi E, Lorenzi C, Lucic S, Fabbri L M, Bertine M and Clini E M. Effectiveness of Erdostine in Elderly Patients with Bronchiectasis and Hypersecretion: a 15 Day, Prospective, Parallel, Open-Label, Pilot Study. Clinical Therapeutics/Vol 29, Number 9, 2007.
Evans D J, Bara A and Greenstone M. Prolonged antibiotics for purulent bronchiectasis in children and adults (Review). Cochrane Database of Systematic Reviews, 2011, Issue 5.
Feldman C., Bronchiectasis: New Approaches to Diagnosis and Management; Clin Chest Med 32, 535-546, 2011.
Franco F, Sheikh A, Greenstone M. Short acting beta2-agonists for bronchiectasis (Review). Cochrane Database of Systematic Reviews, 2008, Issue 3.
Godara N, Godara R, Khullar M. Impact of inhalation therapy on oral health. Lung India, 2011; 28(4); 272-5.
Goldstein M F and Chervinsky, P. Efficacy and safety of doxofylline compared to theophylline in chronic reversible asthma—a double-blind randomized placebo-controlled multicenter clinical trial. Med Sci Monit, 2002; 8(4): CR297-304
Hardin D S (August 2004). GH improves growth and clinical status in children with cystic fibrosis—a review of published studies. Eur. J. Endocrinol. 151 Suppl 1: S81-5.
Kapur N, Bell S, Kolbe J, Chang A B. Inhaled steroids for bronchiectasis. Cochrane Database of Systematic Review 2011, Issue 4.
Lasserson T J, Holt K, Evans D J, Milan S J, Greenstone M. Anticholinergic therapy for bronchiectasis. Cochrane Database Syst Rev. 2011, Issue 6.
Lasserson, T J, Holt K, Greenstone, M. Oral steroids for bronchiectasis (stable and acute exacerbations). Cochrane Database Syst Rev. 2001; (4).
Makker K, Agarwal A, Sharma R. Oxidative stress & male infertility. Indian J. Med. Res. 129 (4): 357-67, 2009.
Martinez-Garcia M, Soler-Cataluna J, Catalan-Serra, P, Roman-Sanchez, P and Tordera M P. Clinical Efficacy and Safety of Budesonide-Formoterol in Non-Cystic Fibrosis Bronchiectasis. Chest, 141, 461-468, 2011.
Mitchell, Richard Sheppard; Kumar, Vinay; Robbins, Stanley L.; Abbas, Abul K.; Fausto, Nelson (2007). Robbins Basic Pathology, Saunders/Elsevier. ISBN 1-4160-2973-7.
Mason R J, Broaddus C V, Martin T, King, Schraufnagel D, Murray J F, Nadel J A. Murray and Nadel's Textbook of Respiratory Disease (4^{th} Edition), Saunders/Elsevier, 2005
Naidich D P, McCauley D I, Khouri N F, Stitik F P, Siegelman S S. Computed tomography of bronchiectasis. J Comput Assist Tomogr. 1982 June; 6(3):437-44.
O'Donnell A E. Bronchiectasis. Chest; 134:815-823, 2008.
O'Malley C A. Infection control in cystic fibrosis: cohorting, cross-contamination, and the respiratory therapist. Respir Care 54 (5): 641-57, 2009
Page P. Doxofylline: A “Novofylline”. Pulmonary Pharmacology & Therapeutics, 2010, 23:231-234.
Pan S H, Canafax D M, Le C T, Cipolle R J, Uden D L and Warwick W J. Bronchodilation from intravenous theophylline in Patients with cystic fibrosis: Results of a blinded placebo-controlled crossover clinical trial. Pedatric Pulmonol. 1989; 6:172-179.
Paloucek F P, Rodvold, K A. Evaluation of theophylline overdoses and toxicities. Annal of Emergency Medicine, 1988; 17: 135-144.
Pizzutto S J, Upham J W, Yerkovich S T, Chang A B. Inhaled non-steroid anti-inflammatories for children and adults with bronchiectasis. Cochrane Database of Systematic Reviews, 2010, Issue 4.
Price D, Musgrave S D, Shepstone L, Hillyer E V, Sims E J, Gilbert R F T, Juniper E F, Ayres J G, Kemp L, Blyth A, Edward M A, F. Wilson, M., Wolfe S, Freeman D, Mugford M, Murdoch J, and Harvey I. Leukotriene Antagonists as First-Line or Add-on Asthma-Controller Therapy. N Engl J Med 2011; 364:1695-1707.
Richter A, Anton S, Koch P, and Dennett S. The Impact of Reducing Dose Frequency on Health Outcomes. Clinical Therapeutics (2003); 25(8):23072335.
Sheilkh A, Nolan D and Greenstone M. Long-acting beta2-agonists for bronchiectasis (Review). Cochrane Database of Systematic Reviews, 2010, Issue 10.
Smith M P. Non-cystic fibrosis bronchiectasis. J R Coll Physcians Ednib 2011:41:132-9.
Sobieraj D M, White C M and Coleman C I. Benefits and risks of adjunctive inhaled corticosteroids in chronic obstructive pulmonary disease: a meta-analysis. Clinical Therapeutics, Vol 30, Num 8, 2008: 1416-1425.
Sullivan P, Bekir S, Jaffar Z, Page C P, Jeffery P, Costello J. Anti-inflammatory effects of low-dose oral theophylline in atopic asthma. Lancet 1994; 343:1006e8.
Subie H A and Fitzgerald D A. Non-cystic fibrosis bronchiectasis. Journal of Paediatrics and Child Health, 2012:48, 382-388.
ten Hacken N H and Kerstjens H; Bronchiectasis. Clinical Evidence, 2011: 08:1507.
Theo-24, package insert. Available at U.S. National Library of Medicine website at http://dailymed.nlm.nih.gov/dailymed
Tsang K W, Tan K C and Ho P L. Inhaled fluticasone in bronchiectasis: a 12 month study. Thorax, 60:239-243, 2005.
Walters E H, Gibson P G, Laserson, T J, Walters J A. Long-acting beta2-agonists for chronic asthma in adults and children where background therapy contains varied or no inhaled corticosteroid. Cochrane Database Syst Rev., 2007 Jan. 24; (1): CD0011385.
Wurzel D, Marchant J M, Yerkovisch S T, Upham J W, Master I B and Chang A B. Short courses of antibiotics for children and adults with bronchiectasis. Cochrane Database of Systematic Reviews, 2011, Issue 6.
Yankaskas J R, Marshall B C, Sufian B, Simon R H, Rodman D. (2004). Cystic fibrosis adult care consensus conference report. Chest, 2004 January: 125(1 Suppl): 1S-39S.
Yipin T. Curative effect analysis of treating bronchiectasis and mucus hypersecretion by Erdosteine. China Modern Medicine, Vol 19, Issue 1, January 2012, Remedies and Clinics.
Yoshikawa H. First-line therapy for theophylline-associated seizures. Acta Neurol Scand 115 (4 Suppl): 57-61, 2007.
Young K, Aspenstrand F, Kolbenstvedt F. HRCT and brochography in the assessment of bronchiectasis. Acta Radiol. 1992; 32:439-441.

### PATENTS

None

What is claimed is:

1. A method for treating a human with a respiratory condition, said method comprising administering to a patient an effective amount of an oral dosage form of pharmaceutical composition comprising doxofylline and erdosteine, said doxofylline and erdosteine in weight proportions ranging respectively from about 5 to 95 to about 95 to 5;
said treatment by said pharmaceutical composition providing a synergistic benefit to said patient on a member of the group consisting of lung function measurements of FEV1, FEC, PEF, MIP, MEP, PaO₂ and PaCO₂.

2. The method of claim 1, wherein said weight proportions of doxofylline and erdosteine are in the range respectively from about 10 to 90 to about 90 to 10.

3. The method of claim 1, wherein said weight proportions of doxofylline and erdosteine are in the range respectively from about 20 to $0 to about 80 to 20.

4. The method of claim 1, wherein said weight proportions of doxofylline and erdosteine are in the range respectively from about 30 to 70 to about 70 to 30.

5. The method of claim 1, wherein said weight proportions of doxofylline and erdosteine are in the range respectively from about 40 to 60 to about 60 to 40.

6. The method of claim 1 wherein said pharmaceutical composition is comprised of doxofylline and erdosteine in a total dosage from about 50 mg to about 1,000 mg.

7. The method of claim 1, wherein said pharmaceutical composition is formulated as a member of the group consisting of a capsule, a powder, a thin film, a caplet and a tablet.

8. The method of claim 1, wherein said pharmaceutical composition is formulated as a solution.

9. The method of claim 1, wherein said composition is formulated as a suspension.

10. The method of claim 1, wherein the respiratory condition being treated is bronchiectasis.

11. An oral dosage form pharmaceutical composition comprising doxofylline and erdosteine in weight proportions in the range respectively from about 10 to 90 to about 90 to 10.

12. The pharmaceutical composition of claim 11 useful for the treatment of bronchiectasis.

13. The pharmaceutical composition of claim 12 providing for a patient to whom it is administered a synergistic benefit on a member of the group consisting of consisting of lung function measurements of FEV1, FEC, PEF, MIP, MEP, PaO₂ and PaCO₂.
